(19) United States
(12) Patent Application Publication
(10) Pub. No.: US 2017/0196950 A1
(43) Pub. Date: Jul. 13, 2017

(54) METHOD FOR PRODUCING AUTOLOGOUS TOLEROGENIC DENDRITIC CELLS (TOLDCS) WITH SPECIFIC ANTIGENS AND THEIR USE IN THE PREPARATION OF A MEDICAMENT USEFUL FOR THE TREATMENT OF SYSTEMIC LUPUS ERYTHEMATOSUS (SLE)

(71) Applicant: PONTIFICIA UNIVERSIDAD CATOLICA DE CHILE, Santiago (CL)

(72) Inventors: Carolina LLANOS MUNOZ, Santiago (CL); Alexis Mikes KALERGIS PARRA, Santiago (CL); Fabian Alejandro VEGA TAPIA, Santiago (CL); Andy lgor TORRES BAEZA, Santiago (CL)

(73) Assignee: PONTIFICIA UNIVERSIDAD CATOLICA DE CHILE, Santiago (CL)

(21) Appl. No.: 15/316,372

(22) PCT Filed: Jun. 5, 2015
(86) PCT No.: PCT/IB2015/054267

§ 371 Date: Dec. 5, 2016

Related U.S. Application Data
(60) Provisional application No. 62/008,319, filed on Jun. 5, 2014.

(51) Int. Cl. A61K 39/00; G01N 33/569; G01N 33/68; C12N 5/0784

(52) U.S. Cl. CPC A61K 39/0008; C12N 5/064; G01N 33/56972; G01N 33/6869; A61K 2039/5154; A61K 2039/577; C12N 2501/2304; C12N 2501/22; C12N 2502/1192; C12N 2501/999; C12N 2501/04; C12N 2500/92; G01N 2333/70578; G01N 2333/70532; G01N 2333/70503; G01N 2333/70539; G01N 2333/5412; G01N 2333/5434

(57) ABSTRACT

The invention relates to a method for producing tolerogenic dendritic cells (tolDCs) with specific antigens, comprising the steps of: (a) culturing precursors of dendritic cells in an animal-serum-free medium, using cytokines, IL-4 and GM-CSF, in order to differentiate same in dendritic cells; (b) producing apoptotic cells; (c) culturing the dendritic cells obtained in step (b) in the presence of compounds having anti-inflammatory activity; (d) co-culturing the dendritic cells from step (d) with the apoptotic cells from step (c), such as to stimulate the endocytosis of the apoptotic cells by the dendritic cells; (e) and, by means of identification based on phenotypic evaluation, determining the production of tolerogenic dendritic cells (tolDCs) with specific antigens. The invention also relates to the tolDC cells produced with said method and to the use of said tolDCs with specific antigens in the production of a drug suitable for the treatment of systemic lupus erythematosus.

### FIELD OF THE INVENTION

The present invention is related with the production of tolerogenic dendritic cells for specific antigens designed for using in autoimmune diseases, in particular for the treatment of Systemic Lupus Erythematosus (SLE).

### STATE OF THE ART

Systemic Lupus Erythematosus (SLE) is a chronic autoimmune disease of unknown etiology affecting mainly women of childbearing age with a prevalence of 124 cases for each 100,000 persons. From a clinical point of view, lupus affects multiple organs, including kidneys, heart, lungs, skin, and musculo-skeletal and hematological systems, among others. It also presents an unpredictable clinical evolution characterized by reactivations or outbreaks alternated with remission periods. Although mortality of SLE has decreased significantly in the last 50 years, a patient developing SLE at 20 years of age has a 15% chance of dying by 35 years of age due to lupus itself or an associated complication. SLE also shows an important aggregated morbidity. For example, one of the most relevant complications in terms of impact in survival and life quality for patients is lupus nephritis (LN) affecting up to a 50% of lupus patients. In spite of important efforts to optimize the treatment of this manifestation, still 10-30% of patients with LN develops terminal renal insufficiency and therefore requires dialysis and/or renal transplant. Likewise, lupus patient have a mortality between 2 and 5 times higher than general population, presenting up to 23% of work incapacity and in the case of women of childbearing age, this is accompanied by difficulties in conceiving and higher morbidity-mortality rates during pregnancy compared to healthy women.

Likewise, FDA approved in 2011 for the first time in 30 years, a new drug (belimumab) for SLE treatment. Nevertheless, this drug and other usual use drugs for this disease, including mycophenolate mofetil (MMF), cyclophosphamide and steroids, are non-curative and immunosuppressive non-specific treatments associated to important complications such as opportunistic infections. Recently, the research team of project ALMS (NCT00377637) studying the use of MMF in lupus nephritis, published results of the maintenance phase of said study, wherein the drug was compared to azathioprine. The study shows that 33.3% of patients using azathioprine and 23.5% of patients using MMF presented serious adverse effects motivating their withdrawal from the study in a 39.6% and 25.2% of patients respectively. Other biological agents approved for use in other autoimmune pathologies such as rituximab, have shown no efficacy in controlled clinical studies for lupus, and just like traditional immunosuppressant, can also contribute to emergence of opportunistic infections. The present invention consists in a procedure to obtain autologous dendritic cells (DCs) specific for antigens from apoptotic cells for immunotherapy for SLE. In a search for prior art, certain documents related with provision and use of dendritic cells for treatment of immunological diseases were found. The closest document to the technology under study is the patent application WO2001085207 wherein the use of apoptotic cells is proposed to decrease the capacity of DC cells for stimulating a cellular response through induction of T cell anergy. Also, it includes diverse alternatives to modulate apoptotic antigen display by DCs and the use of maturating factors for DCs. In this case, the apoptotic cells are the ones exerting the modulating activity in the function of dendritic cells. The relationship of apoptotic cells and Systemic Lupus Erythematosus is not mentioned in the document. In the present invention, the objective of using apoptotic cells is different, since it is not directed per se to direct modulation of the function of DCs and it is not relevant how the apoptotic antigens are presented by DCs that will be able to induce T cell anergy. The objective of using apoptotic cells is to achieve that the DCs of the present invention are specific to only modulate the response of T cells to antigens contained in apoptotic cells, which have a relevant role in the pathology of Systemic Lupus Erythematosus. Furthermore, during the performed experiments, an evaluation was conducted to demonstrate that apoptotic cells would not induce maturation of DCs, i.e., that they would not increase the immunogenic capacity since it was never in the objectives of use that they modulate immunogenicity of DCs. The modulation of the function of DCs is achieved using Rosiglitazone (RGZ) and Dexamethasone (DEXA).

The publication by Carreño et al. “Induction of Tolerogenic Dendritic Cells by NF-κB Blockade and Fcg Receptor Modulation” describes the use of DCs treated with Rosiglitazone and andrographolide for a murine model treatment for multiple sclerosis and suggests that it can be used for other autoimmune diseases such as Lupus, wherein the antigen is unknown. Protocols are described to treat animals directly with these drugs and generating tolerogenic DCs (tolDCs) with Rosiglitazone and Andrographolide. In the present invention, the use of Rosiglitazone and Dexamethasone together is proposed to generate tolerogenic DCs, which is not disclosed in the publication by Carreño et al. Also, said publication proposes to treat mice directly with NF-κB inhibitory drugs, rosiglitazone and andrographolide, not with DCs treated with these drugs, as it is proposed in the present invention. The treatment with treated DCs is made in a murine model of multiple sclerosis. The only mention to the possibility of treating SLE with tolerogenic DCs is subtle and never includes the idea of including apoptotic cells to induce anergy in T lymphocytes recognizing antigens from these cells. Even more, it is specifically mentioned that the Lupus antigen are unknown, and never proposes that apoptotic cells can be used as a source of antigens. Even this is shown as a potential obstacle.

The publication of International Patent Application WO2012160200 proposes the use of steroids and Vitamin D to generate mature DCs with tolerogenic properties. The culture time in steroids is 4 days and it explicitly reads “to obtain mature DC”. Afterwards, these cells are cultured in an environment with pro-inflammatory cytokines, wherein mature DCs are identified by the presence of CD14 on their surface and tolerogenic cells are identified by the expression of MERTK. The present invention proposes to obtain immature DCs (iDCs) using dexamethasone (glucocorticoid) and rosiglitazone (thiazolidinedione), which is mentioned in this patent application and it is not similar to Vitamin D. The method of the present invention is different, since the cells are cultured with rosiglitazone and dexamethasone. In no place in this document is suggested or proposed that DCs should have a given specificity for the treatment with some specific antigen. Even more, in no place is suggested that in the case of SLE the source of antigens could be the use of apoptotic cells. The DCs of the present invention are CD14(−), i.e. it is not expressed and the presence of MERTK is not evaluated, since it is about different products originated from different methodologies.

For the generation of immunotherapy based on DCs, the antigenic specificity is relevant, since one of the features of this kind of therapy is decreasing the response against self-antigens without affecting an effective response against external agents. Nevertheless, one of the main obstacles currently complicating the development of immunotherapy in SLE is the lack of knowledge of the specific autoantigen responsible for the development of the disease. Since patients suffering from Lupus present a deficiency for eliminating cellular debris generated in apoptosis process, is that it has been postulated that these would be a source of autoantigens. Therefore, the present invention proposes the use of apoptotic cells as a source of autoantigens for directing the activity of tolerogenic DCs (tolDCs) exclusively towards auto reactive lymphocytes (FIG. 1).

### DESCRIPTION OF FIGURES

FIG. 1. Phenotypes of dendritic cell. Immature cells, of low immunogenic potential, mature in the presence of pro-inflammatory stimulus such as LPS, acquiring a phenotype with elevated capacity of activating T lymphocytes and increased expression of surface markers CD80, CD83, CD86 and CD40. However, when immature cells receive anti-inflammatory stimulus, they can convert into tolerogenic cells inducing tolerance in T lymphocytes without the potential of turning immunogenic.

FIG. 2. Experimental design for generating immature Dendritic Cells (iDCs) and tolerogenic cells (tol DCs) pulsed with apoptotic cells.

FIG. 3: Generation of apoptotic cells using UV-B irradiation. A: representative dot-plot of lymphocytes non-exposed to UV-B light (base). B: Treated with Staurosporine (Novex, Carlsbed, Calif., USA) (1 μM) for 24 hours (+ control for apoptosis). C: Treated for 50 minutes at 56° C. (+ control necrosis). D: Exposed for 1.5 hours to UV-B radiation. E: Percentage distribution of lymphocytes per cuadrant treated with PI and Anex-V-FITC after 1.5 hours of exposure to UV-B radiation of a control individual.

FIG. 4. Experimental design for determining the capacity of DCs for endocytose apoptotic cells.

FIG. 5: Flow cytometry analysis of incorporation of apoptotic cells into monocyte-derived DCs. A: Auto fluorescence histogram of cells and determination of CD11c+ population. B: Histogram for determining the positive signal for CFSE (FITC+). C: CD11c+ population of a control individual. D: FITC+ population histogram obtained from a CD11c+ population in cells from a control individual.

FIG. 6: Confocal microscopy of apoptotic cell endocytosis by monocyte-derived DCs from a SLE patient in the presence of autologous apoptotic cells. In the left panel of the figure, BODIPY TR Ceramide dyed DCs are observed joining the Golgi apparatus of living cells. The central panel shows apoptotic cells dyed with CFSE. The right panel shows the overlapping of the previous images showing endocytosis of apoptotic cells by DCs (white arrows).

FIG. 7: Expression of surface markers CD40, CD80, CD83, CD86 and HLA-DR in human dendritic cells derived from monocytes treated with S. typhimurium (1 μg/ml) lipopolysaccharide (LPS) and co-cultured with autologous apoptotic cells (12.5 μg/ml DNA content, generated by UV-B radiation) in the presence of RGZ (10 μM) and RGZ+Dexa (1 μM) (n=14 LES patients). The asterisk indicates *P<0.05 (Friedman Test) when comparing stimulation with LPS and DCs treated with RGZ, DEXA, co-cultured with apoptotic cells and challenged with LPS. The line represents the value corresponding to DCs treated with the vehicle. Apocell: apoptotic cells; DEXA: dexamethasone; RGZ: rosiglitazone.

FIG. 8: Secretion profile of cytokines IL-6, and IL-12p70 in the supernatant cultures of SLE patient dendritic cells derived from monocytes treated with S. typhimurium (1 μg/ml) lipopolysaccharide (LPS) and co-cultured with autologous apoptotic cells (12.5 μg/ml DNA, content, generated by UV-B radiation) in the presence of RGZ (10 μM) and Dexa (1 μM). The figure shows a significant decrease in secretion of IL-6 and IL-12p70 using both treatments. IL-6 is expressed as “fold-increase” compared to the condition without treatment.*p<0.05 Wilcoxon Test.

FIG. 9: XTT cell viability assay for SLE patients DCs. The figure shows that cell viability of DCs is not altered in presence of treatment with immunosuppressant drugs (n=6) when compared to non-treated DCs that only received the vehicle (VEH). Apoptotic cells (Apocell) generated by UV-B radiation were employed as a negative control of viability.

FIG. 10: Determination of activation of peripheral blood lymphocytes (PBL) with CD69 and CD71 markers, after being treated with supernatant of DCs cultured in the lab for 24 hours. The figure shows the percentage of CD69+ and CD71+ cells for each condition. n=1 healthy control. Positive control: cells treated for 24 hours with Concanavalin A (Con-A, 10 μL/ml); SN: supernatant; Apocell: Apoptotic cells.

FIG. 11: Experimental design of Mixed Lymphocyte Reaction assay. iDC corresponds to immature Dendritic Cells, mDC corresponds to mature Dendritic Cells, and tolDC corresponds to tolerogenic Dendritic Cells.

FIG. 12: Functional assay of tolDC in a Mixed Lymphocyte Reaction with allogenic T lymphocytes. The expression of CD25 (Figure A) and CD71 (Figure B), and dilution of CFSE probe by proliferation, of CD4+ T lymphocytes the fifth day of co-culture with tolDCs, were quantified. The plots represent the percentages of CD4+CD25+, and CD4+CD71+T lymphocytes the fifth day of co-culture (n=1). αCD3+αCD28 is the positive control of T lymphocyte activation. R+D: dexamethasone and rosiglitazone.

FIG. 13. Functional assay of tolDC in Mixed Lymphocyte Reaction with allogenic T lymphocytes. Expression of CD71 (Figure A), and dilution of CFSE probe by proliferation (Figure B), of CD4+ T lymphocytes the fifth day of co-culture with tolDCs, were quantified. The plots represent the percentages of CD4+ CD71+ T lymphocytes, and proliferating (CD4+ CDFSElow) the fifth day of co-culture. (n=1). UT: untreated. R/D: dexamethasone and rosiglitazone.

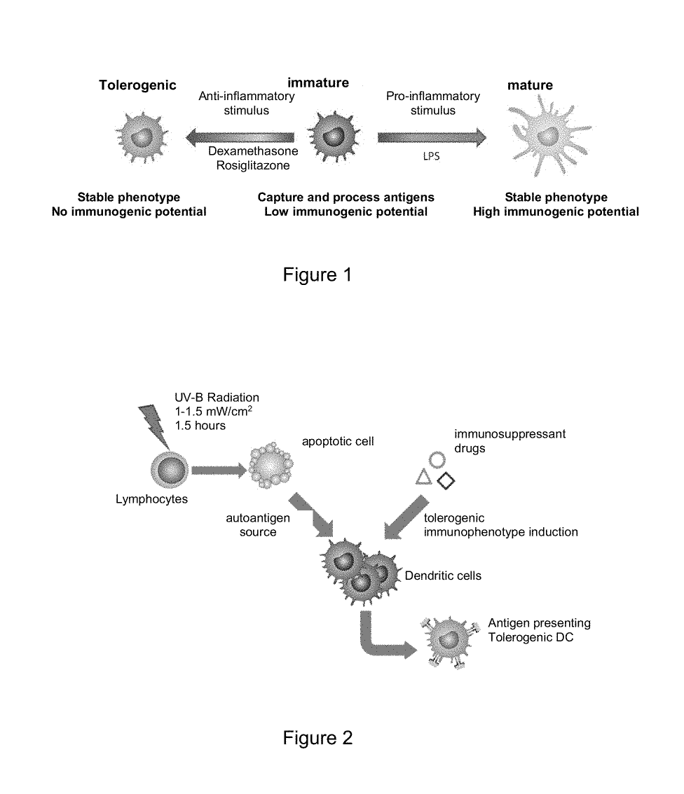

### SUMMARY OF THE INVENTION

The present invention consists in tolerogenic dendritic cells (tolDCs) with specific antigens that reestablish tolerance of immune system to own organs, specific methods that reestablish tolerance of immune system to own organs, method to produce said tolDCs with specific antigens; use of said tolDCs with specific antigens in the production of a therapy for the treatment of Systemic Lupus Erythematosus (SLE).

### DESCRIPTION OF THE INVENTION

The present invention considers three main aspects. In a first aspect, the present invention corresponds to a method to produce tolerogenic dendritic cells (tolDCs) with specific antigens. In a second aspect, the present invention corresponds to tolerogenic dendritic cells (tolDCs) with specific antigens that reestablish the tolerance of the immune system to own organs, when administered to a patient in need thereof. Finally, a third aspect of the present invention corresponds to the use of tolerogenic dendritic cells (tolDCs) with specific antigens in a therapy for Systemic Lupus Erythematosus (SLE).

An embodiment of the first aspect of the invention corresponds to a method to produce tolerogenic dendritic cells (tolDCs) with specific antigens. In a particular embodiment, the method to produce tolerogenic dendritic cells (tolDCs) with specific antigens comprises the following steps:
(a) culturing dendritic cell precursors in vitro in an animal serum free medium, using cytokines IL-4 and GM-CSF, for differentiating them into dendritic cells;
(b) producing apoptotic cells;
(c) culturing dendritic cells obtained in step a) in the presence of compounds with anti-inflammatory activity;
(d) co-culturing dendritic cells of step c) with apoptotic cells from step b), in a manner to propitiate endocytosis of apoptotic cells by dendritic cells;
(e) determining through phenotypic evaluation identification procurement of tolerogenic dendritic cells (tolDCs) with specific antigens.

In a particular embodiment, the dendritic cell precursors of step a) are selected among monocytes, bone marrow progenitors, or directly from peripheral blood or umbilical cord blood.

In another particular embodiment, differentiation is performed when culturing precursors and cytokines IL-4 and GM-CSF in conditions between 30 y 45° C., and between 1 and 10% CO₂. More specifically at 37° C. and 5% CO₂.

In other embodiment, the apoptotic cells of step c) are produced when exposing cells to an apoptotic stimulus selected from ultraviolet B (UV-B) radiation, presence of chemical substances (staurosporine, methotrexate), activation of specific receptors (Fas-Fas ligand interaction) or inhibition of mitochondrial electron transport (heptachlor, rotenone). In another embodiment, the cells from which the apoptotic cells are originated correspond to blood cells, muscular cells, epidermal cells, epithelial cells, stem cells or human cell lines. In a particular embodiment, the blood cells are peripheral blood lymphocytes, platelets, neutrophils, or monocytes. In a more specific embodiment, the blood cells are peripheral blood lymphocytes. In another embodiment, culture of dendritic cells in presence of compounds with anti-inflammatory activity of step d) is performed during a period between 5 and 48 hours. In a more specific embodiment, the compounds with anti-inflammatory activity are selected from rosiglitazone (RGZ) and dexamethasone (DEXA) or a combination thereof. In a more specific embodiment, dendritic cells are cultured in the presence of between 5 and 30 μM RGZ, in presence of between 0.5 and 5 μM de DEXA.

In a specific embodiment, co-culture of dendritic cells of step e) with apoptotic cells is performed considering an amount of apoptotic cells, expressed as DNA content, between 5 and 20 μg/ml. Animal serum-free medium is used to this end, for example AIM-V (GIBCO® AIM V® Medium Grand Island, N.Y., USA).

In other embodiment, co-culture of dendritic cells with apoptotic cells is performed during a period between 5 and 48 hours.

In other embodiment of the invention, identification of tolDCs of step f) is performed by evaluating: i) production of cytokines IL-6, and IL-12p70 which must decrease compared to mature immunogenic DCs; and ii) absence or reduced expression of surface markers compared to mature immunogenic DCs, wherein the surface markers are selected from CD40, CD80, CD83, CD86, HLA-DR, or combinations thereof. In a more specific embodiment, the evaluation of the production of IL-6, and IL-12p70 and expression of surface markers CD40, CD80, CD83, CD86, HLA-DR, or combinations thereof is performed using a technique selected from ELISA, flow cytometry, Western blot, and also level of transcription of messenger RNA through RT-PCR.

A second aspect of the invention, corresponds to tolerogenic dendritic cells (tolDCs) with specific antigens obtained using the previously exposed method. In a specific embodiment, the specific antigens are autoantigens, and not because they come from a patient, since in autoimmune diseases the immunological response is against a self-element, and therefore, the antigen is an autoantigen. In a specific embodiment of the invention, dendritic cells come from: monocytes, bone marrow progenitors, or directly from peripheral blood or umbilical cord blood. In another embodiment, the specific antigens come from apoptotic cells. In a more specific embodiment, the apoptotic cells come from cells that have been subjected to an apoptotic stimulus. In a specific embodiment, the apoptotic stimulus to which the cells are subjected to, is selected from ultraviolet B (UV-B) radiation, in the presence of chemical substances (staurosporine, methotrexate), activation of specific receptors (Fas-Fas ligand interaction) or inhibition of mitochondrial electron transport (heptachlor, rotenone). More specifically, the stimulus is ultraviolet type B (UV-B) radiation.

In other embodiment, the cells from which the apoptotic cells come from, correspond to blood cells, muscular cells, epidermal cells, epithelial cells, stem cells, or human cell lines. In a particular embodiment, the blood cells are peripheral blood cells, platelets, neutrophils, or monocytes.

In a specific embodiment, tolerogenic dendritic cells (tolDCs) of the invention are identified through phenotypic evaluation. In a more specific embodiment, the identification of tolDCs is made by evaluating: i) production of cytokines IL-6, y IL-12p70 which must decrease compared to immunogenic mature DCs; and ii) absence or reduced expression of surface markers compared to mature immunogenic DCs, wherein the surface markers are selected from CD40, CD80, CD83, CD86, HLA-DR, or combinations thereof. In a more specific embodiment, the evaluation of production of IL-6, and IL-12p70 and expression of surface markers CD40, CD80, CD83, CD86, HLA-DR, or combinations thereof, is performed using a technique selected from ELISA, flow cytometry, Western blot, and also transcription level of messenger RNA using RT-PCR.

In a third aspect of the invention, the use of tolerogenic dendritic cells (tolDCs) with specific antigens is described in the production of a therapy for the treatment of Systemic Lupus Erythematous (SLE).

In a specific embodiment, the invention describes the use of tolerogenic dendritic cells (tolDCs) with specific antigens, which can be used in the preparation of a medicine useful in the treatment of Systemic Lupus Erythematous (SLE).

### EXAMPLES OF APPLICATION

### Example 1: Obtaining DCs Specific for Antigens Relevant in SLE

Apoptosis Induction Using Ultraviolet Type 8 (UV-8) Radiation in Peripheral Blood Lympchotytes

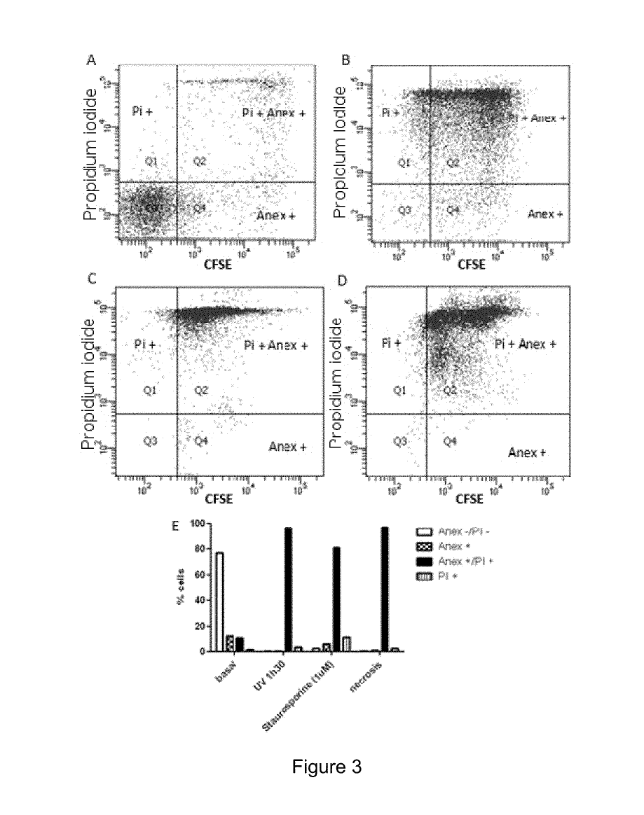

In this stage, different exposition times to UV-B radiation were evaluated to obtain apoptotic cells from lymphocytes from peripheral blood from initially healthy individuals and afterwards from patients with SLE. To corroborate the apoptosis induction, Propidium Iodide (PI) and Annexin V (Anex) were used as dyes and the samples were analyzed using flow cytometry. Said cells in apoptosis status are later used to pulse DCs with the aim to provide autoantigens (FIG. 2). FIG. 3 shows that an exposition time of 1.5 hours to UV-B radiation is suitable to induce apoptosis (PI+Anex+) in these cells.

Evaluation of the Capacity of DCs of Patients with SLE to Endocytose Apoptotic Cells.

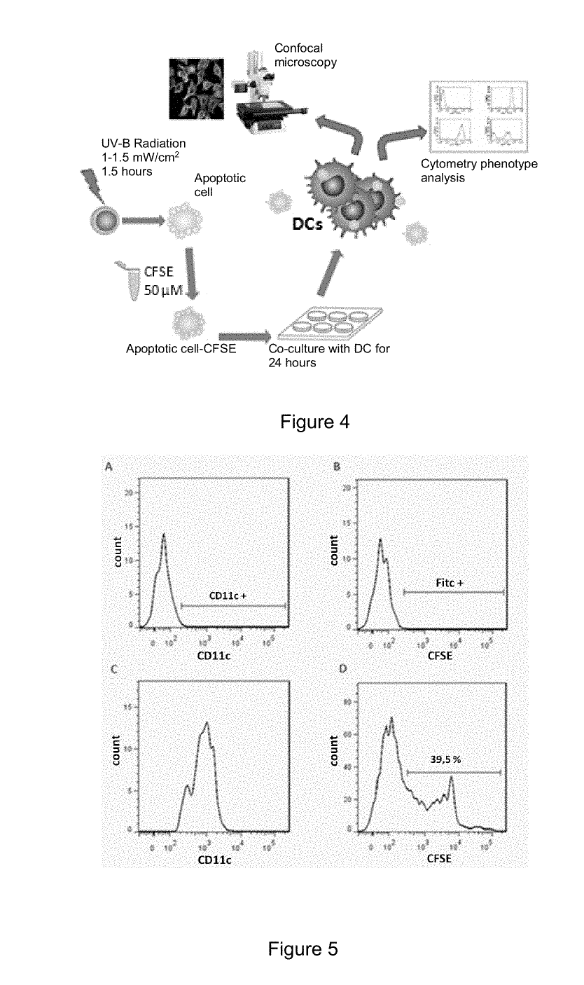

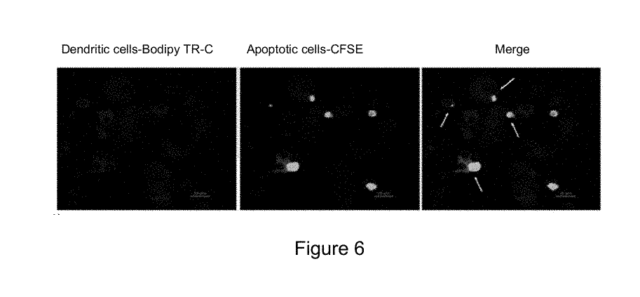

Afterwards, it was evaluated if DCs generated in the previous Example had the capacity to endocytose apoptotic cells to process antigens and then present them to self-reactive T lymphocytes. Apoptotic cells generated through exposition to UV-B radiation were labeled with cellular dye carboxyfluorescein succinimidyl ester (CFSE) (Invitrogen, Carlsbad, Calif., USA) to detect them using flow cytometry. DCs were incubated for 24 hours with apoptotic cells labeled with CFSE. This experiment selected a population CD11c+ allowing to identify DCs. CFSE detection in a CD11c+ population (39.5%) is indicative of phagocytosis of apoptotic cells by DCs (FIGS. 4 and 5). Briefly, apoptotic cells generated by UV-B radiation were labeled with CFSE and co-cultured for 24 hours with DCs. DCs were labeled with vital dye BODIPY-TR Ceramide (Life Technologies), and as observed in FIG. 6, the composition of the image shows that DCs are able to endocytose apoptotic cells in a control individual (panel B) as well as SLE patients (panel C).

Efecto de Co-Cultivo De Células Apoptóticas y DCs en Inmunofenotipo de DCs.

It is widely reported that DNA molecules play a role as autoantigen in lupus patients and that are exposed to extracellular space during apoptosis associated to proteins, such as histones in the form of nucleosomes (1). Therefore, determining DNA constitutes a manner to quantify apoptotic cells, a method previously used by other researchers (2).

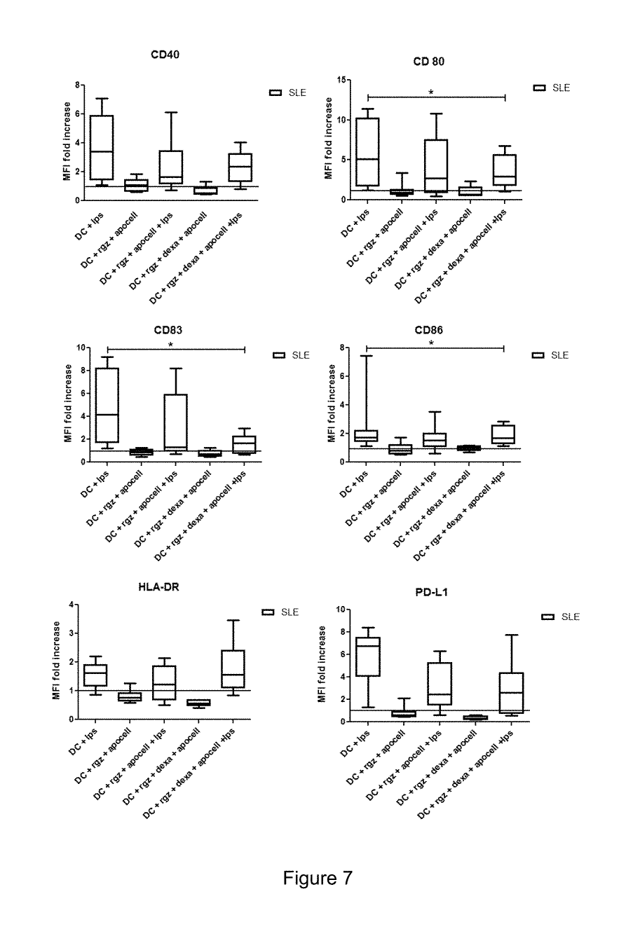

For the results shown in the present invention, a total of 14 SLE patients were included, whose clinical characteristics are detailed in Table 1, from whom DCs were generated and the sixth day were pulsed with apoptotic cells (12.5 μg/ml of DNA content) for 24 hours prior to the analysis. The maturation state of resulting DCs was analyzed by flow cytometry using conjugated antibodies anti CD40, CD80, CD83, CD86 and HLA-DR (FIG. 7).

TABLE 1
Clinical characteristics of SLE patients for the study of DC generation and co-culture with apoptotic cells. The activity
degree of the disease for each patient included in the study was determined using SLEDAI index, a higher score, higher activity,
defining an active disease as having SLEDAI >6. Also included is the average age and SLE criteria for each patient.

| Patient | Gender | Age (yrs) | SLEDAI | Treatment | Arthritis | Immune | NS | Kidney | Haem. | Serositis | MC | ANA |
|---|---|---|---|---|---|---|---|---|---|---|---|---|
| SLE6 | F | 30 | 10 | None | + | + | − | + | + | + | + | + |
| SLE7 | F | 46 | 4 | PDN 5 Mg | − | + | − | − | + | − | + | + |
| SLE8 | F | 25 | 14 | HCQ, PDN 15 Mg | + | + | − | − | − | − | + | + |
| SLE9 | M | 23 | 8 | HCQ, PDN 20 Mg, MMF | + | + | − | + | − | − | − | + |
| SLE10 | F | 34 | 2 | HCQ, PDN 5 Mg, MMF | + | + | − | + | + | − | + | + |
| SLE11 | F | 65 | 0 | HCQ, PDN 5 Mg | − | + | − | + | + | − | − | + |
| SLE12 | M | 24 | 2 | HCQ, MMF | + | + | − | + | − | − | − | + |
| SLE13 | F | 24 | 0 | HCQ | + | − | − | − | + | − | + | + |
| SLE14 | F | 26 | 2 | HCQ | + | + | − | + | − | − | + | + |
| SLE15 | F | 36 | 0 | HCQ | + | + | − | − | + | − | − | + |
| SLE16 | F | 35 | 8 | HCQ | − | + | − | − | + | − | + | + |
| SLE17 | F | 26 | 6 | HCQ, PDN 10 Mg, CYT | − | + | − | − | + | − | + | + |
| SLE18 | F | 47 | 2 | HCQ, PDN 5 Mg, AZT | − | − | − | − | − | + | + | + |
| SLE19 | F | 49 | 4 | HCQ, PDN 10 Mg, AZT | + | + | − | − | − | − | + | + |
| Average | | 35 ± 12.5 | 4 ± 4.2 | | | | | | | | | |
| (±SD) | | | | | | | | | | | | |

Art. Arthritis; Imm. = immunological (presence of antibodies anti-DNA, anti-Sm, or anti-cardiolipins); SN: nervous system compromise (convulsions or psychosis); Hem. = hematological compromise; Sero. = serositis; MC = mucocutaneous; ANA: anti-nuclear antibodies.

RGZ and DEXA Effect on DC Pulsed with Apoptotic Cells.

DCs derived from monocytes obtained from SLE patients and control individuals were treated for 24 hours with RGZ (10 μM) and DEXA (1 μM) and then co-cultured with apoptotic cells (12.5 μg/ml of DNA content) for 24 additional hours and the expression of markers CD40, CD80, CD83, CD86 and HLA-DR was evaluated in a total of 14 SLEpatients (Table 1). FIG. 7 shows that DC in presence of apoptotic cells do not present variation of any type of the studied maturation markers, while the treatment with drugs RGZ+DEXA significantly reduces (p<0.05, Friedman Test) the expression of maturation markers, CD80, CD83 and CD86 in DCs from SLE patients, co-cultured with apoptotic cells compared to cells treated with S. typhimurium LPS, demonstrating the effectivity of the treatment with immunosuppressant drugs in the change of DCs phenotype.

Determination of Activation of DCs: Proinflammatory and Antiinflammatory Cytokines Secretion.

With the purpose to corroborate the induction of a tolerogenic state in DCs and having more elements allowing the most complete characterization possible and a functional approach, a quantification of some relevant cytokines in supernatants of previous experimental supernatants was performed, such as interleukin 6 (IL-6) and interleukin 12p70 (IL-12p70), secreted to extracellular medium by tolerogenic dendritic cells, using Enzyme-Linked Immunosorbent Assay (ELISA).

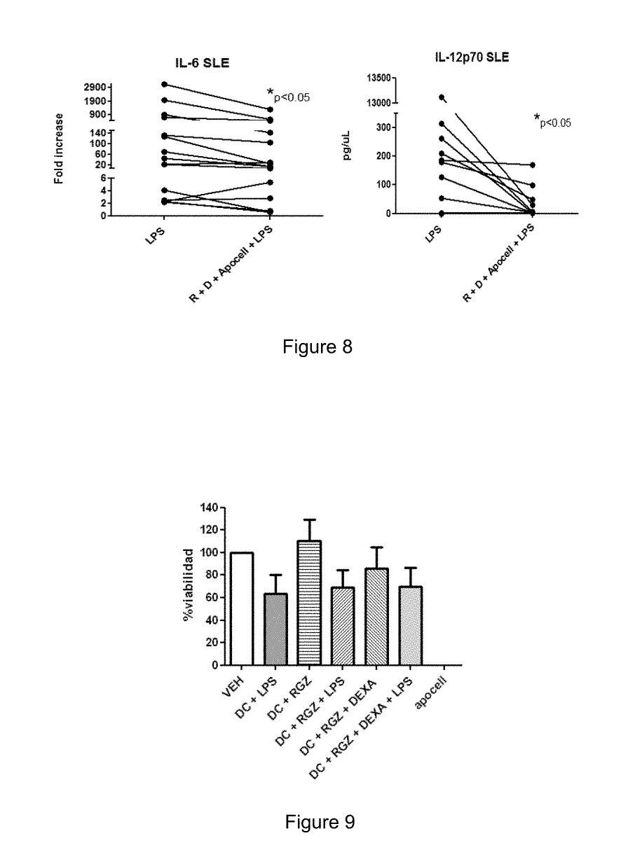

FIG. 8 shows the treatment with RGZ+DEXA inducing a significant decrease (p<0.05) in secretion of IL-6, a multifunctional cytokine with clearly defined properties in the regulation of inflammatory states (3) and during antigen display plays a fundamental role in activation and differentiation of CD4+ T lymphocytes to some of the effector phenotypes (4), compared to DCs treated with S. typhimurium LPS. In the same figure is also observed a decrease in secretion of IL-12p70, this cytokine plays an important role in potentiating cytotoxic activity of Natural Killer (NK) cells as well as cytotoxic activity of CD8+ T lymphocytes and the development of a pro-inflammatory phenotype in CD4+ T lymphocytes.

Table 2 summarizes the obtained results and shows that the product tolDC (tolerogenic DCs treated with RGZ and DEXA and pulsed with apoptotic cells) presents a decrease in most of the relevant surface markers associated to maturity and an immunogenic phenotype when challenged with LPS and a decrease in production of pro-inflammatory cytokines when compared to DC that were not treated with RGZ and DEXA.

TABLE 2
Summary of phenotype observed in mature DCs and toIDCs.

| | Phenotype markers | | | | | Cytokines | |
|---|---|---|---|---|---|---|---|
| | | | | | HLA- | | IL- |
| | CD40 | CD80 | CD83 | CD86 | DR | IL-6 | 12p70 |
| Immunogenic | ↑ | = | ↑ | = | = | ↑ | ↑ |
| DCs (DCs + | | | | | | | |
| LPS) | | | | | | | |
| tolDCs (DCs + | = | ↓ | ↓ | ↓ | = | ↓ | ↓ |
| R + D + | | | | | | | |
| Apocell + LPS) | | | | | | | |

Toxicity and Cell Viability Studies for SLE Therapy with Autologous Tolerogenic DCs.

With the objective to evaluate the effects of the drugs on viability and cell metabolism, an XTT viability assay was performed, a colorimetric assay estimating the metabolic cellular activity. Its basis consists in reduction of tetrazolium XTT salt, which is transformed to formazan due to the activity of mitochondrial dehydrogenase enzymatic activity of metabolically active cells, generating as a product a colored compound, which is only generated in viable cells and which amount is produced in a proportional amount to the number of viable cells in the sample.

FIG. 9 shows that the treatment with the drugs does not alter the percentage of cell viability of DCs of SLE patients.

Following the same previous line, an experiment was performed to evaluate if generated DCs secreted any potentially toxic substance to extracellular medium which could affect viability of the cells of the organism that would receive the therapy. To perform this assay, propidium iodide (PI) was used together with Annexin V (Anex V) which were used to label peripheral blood lymphocytes (PBL) of a control individual that were previously treated with the supernatants of DCs cultured in the conditions of our protocol using immunomodulating drugs, but without subjecting them to LPS challenge, since they came from another control individual, during a period of 24 hours.

On the other hand, it was determined if these supernatants from generated DCs were able to produce in the lymphocytes an activation response, and for that, markers CD69 (early activation marker) and CD71 (late activation marker) were determined, which are expressed in response to activation stimulus in lymphoid cell lines.

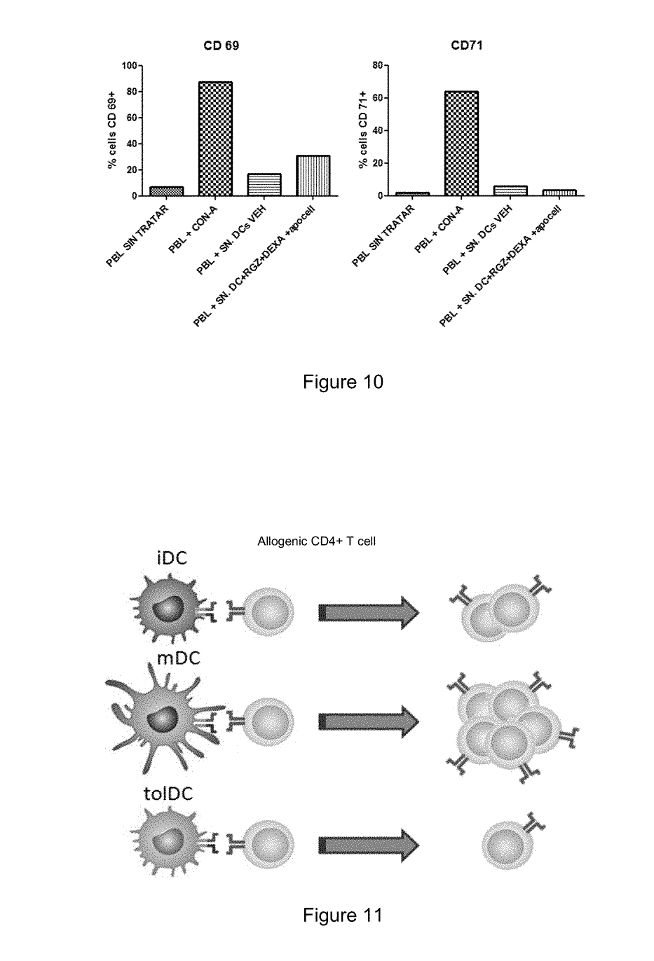

FIG. 10 shows that marker CD71 does not vary its expression when treated with supernatant of DCs treated with the vehicle, and neither the supernatant of DCs treated with drugs and apoptotic cells. Regarding marker CD69, a small increase in percentage of activated cells is observed, but that is far from the levels of activation of a positive control corresponding to PBL stimulated with concanavalin A (a protein with the ability to induce mitogenic activity in T lymphocytes and increase synthesis of cellular products) and that it is similar to the results obtained with supernatants from DCs treated with vehicle. From this two results it is concluded that the supernatants of DCs do not alter the viability of PBL, as they neither generated an activation response in these cells.

### Example 2: Functional Evaluation of Tolerogenic Capacity of DCs

In the previous example, the qualities and stability of the immunophenotype of tolDCs was characterized as a measure to evaluate their potential as therapy and having a functional approach when studying cytokine secretion. To determine their success in a pre-clinical phase, in vitro functional analyses as such are required directed to evaluate the modulating capacity of generated tolDCs on CD4+ T lymphocytes.

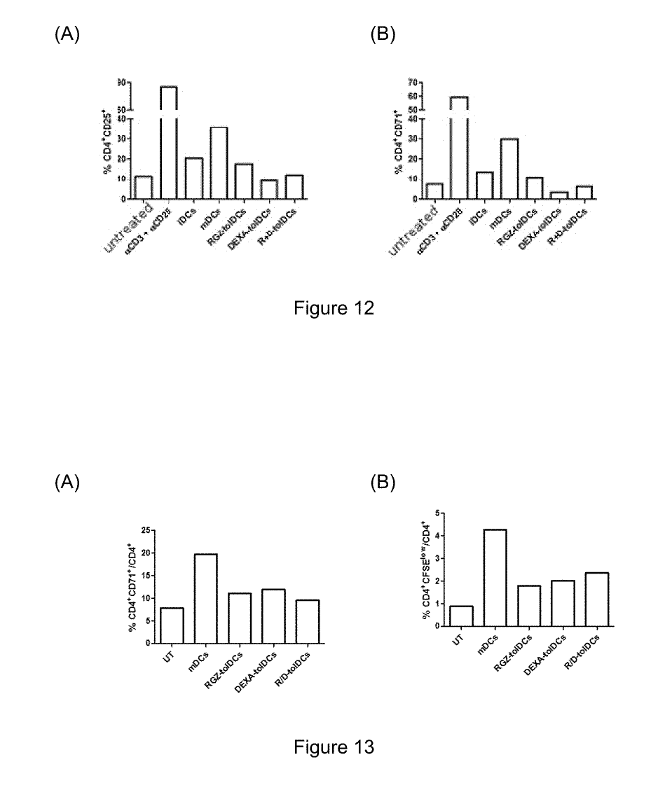

Therefore, a Mixed Lymphocyte Reaction (MLR) allogenic assay was performed using DCs and CD4+ T cells from different healthy individuals (FIGS. 11 and 12).

tolDCs from an individual and T cells from a different individual previously labeled with CFSE were cultured in 200 μL of medium RPMI 1640+10% FBS (ratio 1:5) for 5 days. As a control, T lymphocytes co-cultured with immature DCs (iDCs), mature DCs or without DCs were used until the endpoint of the experiment. The activation of T lymphocytes was evaluated at the fifth day using flow cytometry controlling the expression of CD25 and CD71 (FIG. 12). The expression of CD25 and CD71 (T lymphocyte activation markers) of T cells cultured in the presence of tolDCs induced for each one of the drugs or both drugs simultaneously, is inferior to the one observed for cells co-cultured with immature DCs. The results show the tolerogenic function of DCs induced by the treatment with NF-κB inhibiting drugs.

The same assay was performed with tolDCs from a SLE patient and T cells from a healthy individual previously labeled with CFSE and cultured in 200 μL of medium RPMI 1640+10% FBS (ratio 1:5) for 5 days. As a control, T lymphocytes co-cultured with immature DCs, mature DCs or without DCs were used until the endpoint of the experiment. Proliferation and activation of T lymphocytes on the fifth day were evaluated using flow cytometry by measuring CFSE dilution and CD71 expression (FIG. 13).

The expression of CD71 (T lymphocyte activation marker) in T cells cultured in the presence of tolDCs induced for each one of the drugs or both simultaneously, is inferior than the one observed for cells cultured with immature DCs. Additionally, it was observed that T lymphocytes proliferate less in the presence of tolDCs than mature DCs.

### Example 3: tolDCs Generation Protocol in Systemic Lupus Erythematous

A. Isolation of Peripheral Blood or Buffy Coat should not be Greater than 8 Hours and Supplemented with Heparin
1. Blood was distributed in 50 ml conic tubes and diluted using PBS 1× until reaching a total volume of 35 ml.
2. 15 ml Ficoll-Paque lymphocyte separation medium was added to the bottom of the empty 50 ml conic tube.
3. Diluted blood was carefully transferred to each 50 ml tube containing the Ficoll-Paque medium.
4. This was centrifuged at 1000×g for 25 minutes at 20° C.
5. The upper layer is aspirated (serum) leaving the mononuclear cell layer unaltered in the interphase.
6. The mononuclear cell layer was carefully transferred to a new 50 ml conic tube.
7. The 50 ml conic tube containing the mononuclear cell layer was filled with PBS 1× and was centrifuged at 300×g at 20° C. for 10 minutes. The supernatant was carefully removed and discarded.
8. The pellet was resuspended in 5 ml of RBC lysis buffer (ACK 1×) for 5 minutes at room temperature.
9. The 50 ml conic tube was filled with PBS 1× and mixed carefully.
10. It was centrifuged at 300×g for 10 minutes and the supernatant was discarded.
11. Steps 7, 8 and 9 were repeated once.
12. The pellet was resuspended in 5 ml of pre-heated medium AIM-V. A cell count was performed.
13. Cells were plated in 6-well plated at 10×10⁶ PBMCs in 1 mL of medium AIM-V.
14. The incubation was for 2 hours at 37° C., 5% CO₂ and then section B followed.

B. Obtaining Peripheral Blood Lymphocytes and Differentiation Towards a DC.
1. The supernatant was carefully removed to take the peripheral blood lymphocytes (PBL) without touching the bottom of the tube. (PBL were stored until generation of apoptotic cells). It was washed 3 times with 1 ml PBS 1× preheated at 37° C.
2. 1.5 mL of preheated AIM-V medium were added, containing IL-4 (1000 Ul/mL) and GM-CSF (1000 Ul/mL) (Day 1). It was incubated at 37° C., 5% CO₂
3. Fresh cytokines were added (IL-4 and GM-CSF) on days 3 and 5, until a final concentration (1000 Ul/mL) in the culture without changing the medium.

C. Apoptotic Cell Generation.
1. The non-adherent fraction (PBL) was transferred to a 50 mL conic tube and was filled with PBS 1×.
2. It was centrifuged at 300×g for 6 minutes.
3. It was resuspended in 5 mL of AIM-V medium and was plated in a T-25 culture flask at 37° C. with 5% CO₂. Medium AIM-V was changed daily.
4. An UV lamp was mounted inside the biosafety cabinet and the lamp was preheated for 10 minutes.
5. Carefully PBL were transferred to a sterile plate of 60×15 ml.
6. Lymphocytes were irradiated for 1.5 hours at 2.0 mW/cm². Section D followed.

D. Co-Culture of DCs and Apoptotic Cells (Day 7)
1. Apoptotic cells were homogenized and the final volume was determined after the UV treatment. DNA concentration was determined using 400 μL of the preparation of apoptotic cells.
2. Cells were transferred to a conic tube and centrifuged at 500×g for 10 minutes.
3. The supernatant was carefully discarded and the pellet was resuspended to a final DNA concentration of 1 μg/mL.
4. 18.75 μL of the apoptotic cell preparation were added to the culture medium of DC prepared in section A.

E. Induction of Tolerogenic DC (Day 6)
1. Rosiglitazone was dissolved in DMSO to prepare a stock solution (100 μL DMSO/1.79 mg rosiglitazone). 5 μL of stock solution were diluted with 495 μL PBS 1× to prepare a working solution. 30 μL of the working solution were added to the DC cell culture (final concentration=10 μM).
2. Dexamethasone was dissolved in DMSO to prepare a stock solution (100 μL DMSO/0.2 mg dexamethasone). 5 μL of stock solution were diluted with 20 μL PBS 1× to prepare a working solution. 1.5 μL of working solution were added to the DC cell culture medium (final concentration=1 μM).

### REFERENCES

(1) Rosen A, Casciola-Rosen L. Autoantigens in systemic autoimmunity: critical partner in pathogenesis. Journal of internal medicine. June 2009; 265(6):625-631.
(2) Fehr E M, Spoerl S, Heyder P, et al. Apoptotic-cell-derived membrane vesicles induce an alternative maturation of human dendritic cells which is disturbed in SLE. J Autoimmun. February 2013; 40:86-95.
(3) Wolf J, Rose-John S, Garbers C. Interleukin-6 and its receptors: a highly regulated and dynamic system. Cytokine. 2014 November; 70(1):11-20. doi: 10.1016/j.cyto.2014.05.024. Epub 2014 Jun. 28. Review. PubMed PMID: 24986424.
(4) Zhu J, Paul W. CD4 T cells: Fates, functions and faults. Blood, 2008; 112(5):1557-1569.
(5) Nagy Z S, Czimmerer Z, Szanto A, Nagy L. Pro-inflammatory cytokines negatively regulate PPARγ mediated gene expression in both human and murine macrophages via multiple mechanisms. Immunobiology. 2013 November; 218(11):1336-44. doi: 10.1016/j.imbio.2013.06.011. Epub 2013 Jul. 1. PubMed PMID: 23870825.
(6) Hontelez S, Karthaus N, Looman M W, Ansems M, Adema G J. DC-SCRIPT regulates glucocorticoid receptor function and expression of its target GILZ in dendritic cells. J Immunol. 2013 Apr. 1; 190(7):3172-9. doi: 10.4049/jimmunol. 1201776. Epub 2013 Feb. 25. PubMed PMID: 23440419.

1. A method to produce tolerogenic dendritic cells (tolDCs) with specific antigens, comprising the following steps:
(a) culturing dendritic cell precursors in an animal serum free medium, using cytokines IL-4 and GM-CSF, to differentiate into dendritic cells;
(b) producing apoptotic cells;
(c) culturing dendritic cells obtained in step a) in the presence of compounds with anti-inflammatory activity;
(d) co-culturing dendritic cells of step c) with apoptotic cells of step b), in order to promote endocytosis of apoptotic cells by dendritic cells;
(e) determining through identification by phenotype identification that tolerogenic dendritic cells (tolDCs) with specific antigens were obtained.

2. The method of claim 1, wherein the dendritic cell precursors of step a) are selected from monocytes, bone marrow progenitors, or directly from peripheral blood or umbilical cord blood.

3. The method of claim 1, wherein differentiation is performed when culturing precursors and cytokines IL-4 and GM-CSF under conditions of between 30 y 45° C., and between 1 y 10% CO₂.

4. The method of claim 1, wherein the apoptotic cells of step c) are produced exposing the cells to an apoptotic stimulus selected from ultraviolet B (UV-B) radiation, presence of chemical substances selected from staurosporine or methotrexate, activation of specific receptors such as FAS-FAS ligand interaction, or inhibition of mitochondrial electron transport with heptachlor or rotenone.

5. The method of claim 1, wherein the cells from which apoptotic cells come from are blood cells, muscular cells, epidermal cells, epithelial cells, stem cells, or human cell lines.

6. The method of claim 5, wherein blood cells are peripheral blood lymphocytes, platelets, neutrophils, or monocytes.

7. The method of claim 1, wherein the culture of dendritic cells in presence of compounds with anti-inflammatory activity of step d) is performed for a period between 5 and 48 hours.

8. The method of claim 7, wherein the compounds with anti-inflammatory activity are selected from rosiglitazone (RZG), dexamethasone (DEXA) or a combination thereof.

9. The method of claim 8, wherein the dendritic cells are cultured in the presence of between 5 and 30 μM of rosiglitazone, and in the presence of between 0.5 and 5 μM of dexamethasone.

10. The method of claim 1, wherein the co-culture of dendritic cells of step e) with apoptotic cells is performed considering an amount of apoptotic cells, expressed as DNA content, between 5 and 20 μg/ml.

11. The method of claim 10, wherein the co-culture is made in an animal serum free medium.

12. The method of claim 1, wherein the co-culture of dendritic cells with apoptotic cells is made for a period of time between 5 and 48 hours.

13. The method of claim 1, wherein in step e identification of tolDCs is made by evaluating: i) production of cytokines IL-6, and IL-12p70 which must be lower compared to immunogenic mature DCs; and ii) absence or reduced expression of surface markers compared to immunogenic mature DCs, wherein the surface markers are selected from CD40, CD80, CD83, CD86, HLA-DR, or combinations thereof.

14. The method of claim 13, wherein the evaluation of production of IL-6, and IL-12p70 and the expression of surface markers CD40, CD80, CD83, CD86, HLA-DR, or combinations thereof is made using a technique selected from ELISA, flow cytometry, Western blot, and level of transcription or messenger RNA using RT-PCR.

15. Tolerogenic dendritic cells (tolDCs) with specific antigens wherein tolDCs are obtained using the method of claim 1.

16. Tolerogenic dendritic cells (tolDCs) with specific antigens of claim 15 wherein they come from: monocytes, bone marrow progenitors, or directly from peripheral blood or umbilical cord blood.

17. Tolerogenic dendritic cells (tolDCs) with specific antigens of claim 15 wherein the specific antigens are autoantigens.

18. Tolerogenic dendritic cells (tolDCs) with specific antigens of claim 15 wherein the specific antigens come from apoptotic cells.

19. Tolerogenic dendritic cells (tolDCs) with specific antigens of claim 15 wherein the apoptotic cells come from cells that have been subjected to an apoptotic stimulus.

20. Tolerogenic dendritic cells (tolDCs) with specific antigens of claim 19 wherein the apoptotic stimulus to which the cells are subjected is selected from ultraviolet type B (UV-B) radiation; presence of chemical substances selected from staurosporine or methotrexate; activation of specific receptors such as FAS-Fas ligand interaction; or inhibition of the mitochondrial electron transport using heptachlor or rotenone.

21. Tolerogenic dendritic cells (tolDCs) with specific antigens of claim 19 wherein apoptotic cells come from blood cells, muscular cells, epidermal cells, epithelial cells, stem cells, or human cell lines.

22. Tolerogenic dendritic cells (tolDCs) with specific antigens of claim 21 wherein the blood cells are peripheral blood lymphocytes, platelets, neutrophils, or monocytes.

23. Tolerogenic dendritic cells (tolDCs) with specific antigens of claim 15 wherein the cells are identified using phenotype evaluation.

24. Tolerogenic dendritic cells (tolDCs) with specific antigens of claim 23 wherein the identification of tolDCs is made evaluating: i) cytokine production IL-6, and IL-12p70 which must be lower compared to immunogenic mature DCs; and ii) absence or reduced expression of surface markers compared to immunogenic mature DCs, wherein the surface markers are selected from CD40, CD80, CD83, CD86, HLA-DR, or combinations thereof.

25. Tolerogenic dendritic cells (tolDCs) with specific antigens of claim 24 wherein the evaluation of production of IL-6, and IL-12p70 and the expression of surface markers CD40, CD80, CD83, CD86, HLA-DR, or combinations thereof is made using a technique selected from ELISA, flow cytometry, Western blot and transcription level or messenger RNA using RT-PCR.

26. A method for treating Systemic Lupus Erythematous (SLE) comprising administering tolerogenic dendritic cells (tolDCs) with specific antigens according to claim 15 to a patient in need thereof.

27. (canceled)
